(19) United States
(12) Patent Application Publication
(10) Pub. No.: US 2006/0000120 A1
(43) Pub. Date: Jan. 5, 2006

(54) Proprioceptive orthopedic sole comprising modular correction means

(76) Inventors: Pascal Chenut, Semezanges (FR); Bernard Deren, Ahuy (FR); Jean Douhaire, Dijon (FR)

Correspondence Address: YOUNG & THOMPSON, 745 SOUTH 23RD STREET, 2ND FLOOR, ARLINGTON, VA, 22202, US

(21) Appl. No.: 10/511,166

(22) PCT Filed: Apr. 15, 2003
(86) PCT No.: PCT/FR03/01201

§ 371 Date: Jul. 13, 2005

(30) Foreign Application Priority Data
Apr. 15, 2002 (FR) 0204674

(51) Int. Cl. A61F 5/14

(52) U.S. Cl. 36/144

(57) ABSTRACT

A modular proprioceptive orthopedic sole for re-establishing and/or preserving the correct dynamics of running or walking. The sole is made of an elastic material, and includes elements (1) on the upper surface thereof for activating the articular receptors disposed between the astragalus and the calcaneum as soon as a step is habitually initiated by the heel, and elements (3,4,5) guiding the foot when it becomes engaged on the physiological axis of walking; and on the lower surface thereof at least one removable correction element (11,23,13,14) which can result in an abduction or adduction when a deficient or too large step occurs and/or which can correct a varus, valgus, supination and/or pronation.

### TECHNICAL FIELD

The present invention pertains to a proprioceptive orthopedic insole to re-establish and/or preserve the correct dynamics of running or walking, comprising modular means for correcting a varus, valgus, abduction and/or adduction.

In the area of insoles intended to be placed inside shoes, so-called proprioceptive insoles are known intended to re-establish and/or preserve the correct dynamics of walking; this is the case for example in French patent FR 2676918 by the same Registrant. The insole of the invention comprises means for activating the myo-articular receptors located between the talus and the calcaneus as soon as a step is habitually initiated by the heel, the muscular tonus prompted by this initial impulse then being channeled by means guiding the foot over the physiological axis of walking. For this purpose the insole on its upper surface particularly comprises a console, having an upper part with a straight section and whose thickness increases from the heel as far as an area located substantially perpendicular to the neck of the talus, and also comprises lateral sub-cuboid and sub-scaphoid compensating and prompting means to counter varus or valgus torsion, and axial mediotarsal means intended to distribute bearing under the metatarsal regions.

The insoles of the prior art which are exclusively intended to make walking more comfortable are inoperative if there is a major bearing defect in the three spatial planes, exceeding the proprioceptive action of the insole, when practicing sport such as running for example. This bearing defect causes a varus or supination, valgus or pronation, abduction or adduction of the foot leading to joint, tendon and muscle unbalance, the source of numerous types of pain felt when practicing sport.

In this respect, orthopedic insoles are already known that are particularly intended for sports use and comprise means for correcting a varus, valgus, abduction or adduction; this is the case for example in American patent U.S. Pat. No. 4,841,648 describing a modular orthopedic insole. The insole, made in a resilient material, comprises several corrective zones on its upper surface which comprise the loops of a fixation device of “Velcro” type (registered trademark) onto which the corrective elements are attached which are also made in a resilient material and whose undersurface is provided with hooks able to cooperate with the loops of the correction zones. The various corrective elements are advantageously referenced by a color code, a number code or a label whose properties are recalled in a leaflet enclosed with the insole and enabling the user to position one or more corrective elements on the insole according to the pain felt when practicing sport.

The corrective elements of this type of orthopedic insole have the disadvantage of sliding over the upper surface of the insole when the user is running for example, so that said elements soon become ineffective. When practicing sport, foot perspiration combined with body heat and the shear stresses applied by the foot to the corrective elements generate the displacement of these elements making them inoperative or even dangerous by causing foot bearing defects which may lead to muscle, tendon or bone trauma.

One of the purposes of the invention is to overcome all these disadvantages by proposing a proprioreceptive orthopedic insole to re-establish the correct dynamics of walking or running, which comprises modular means for correcting a foot varus, valgus, abduction or adduction.

For this purpose and in accordance with the invention, a modular proprioceptive orthopedic insole is proposed to re-establish and/or preserve the correct dynamics of walking or running; this insole, made in an elastic material, is remarkable in that firstly, on its upper surface, it comprises means for activating the articular receptors located between the talus and the calcaneus as soon as a step is habitually initiated by the heel with means for guiding the foot when it becomes engaged on the physiological axis of walking, and secondly on its undersurface it comprises at least one removable corrective element able to provide abduction or adduction for respective deficient or excessive step initiation, and/or is able to correct a varus or supination and/or valgus or pronation.

It was found, in surprising manner, that the combination of the corrective elements positioned on the undersurface with the means positioned on the upper surface of the insole activating the articular receptors located between the talus and the calcaneus and guiding the foot over the physiological axis of walking, provides improved correction of a valgus and/or varus and/or foot abduction and/or adduction while having recourse to corrective elements of narrow thickness thereby reducing the impression of discomfort of the orthopedic insoles of the prior art.

In particularly advantageous manner, the insole comprises on its undersurface at least one recess positioned along the inner and/or outer edge of said insole, in which the removable corrective element made in a more rigid material than the insole material, and whose shape corresponds to the shape of the recess, is able to be positioned such that the corrective elements remain in place under the insole when running or walking. Also, it will be noted that since the corrective elements are placed on the undersurface of the insole, they do not come into contact with the foot thereby avoiding any plantar skin lesion such as cuts, blisters, fissures or analogue unlike the insoles of the prior art.

Other advantages and characteristics will become more readily apparent in the following description of various embodiments which are given as non-restrictive examples of the proprioceptive orthopedic insole of the invention with reference to the appended drawings in which:

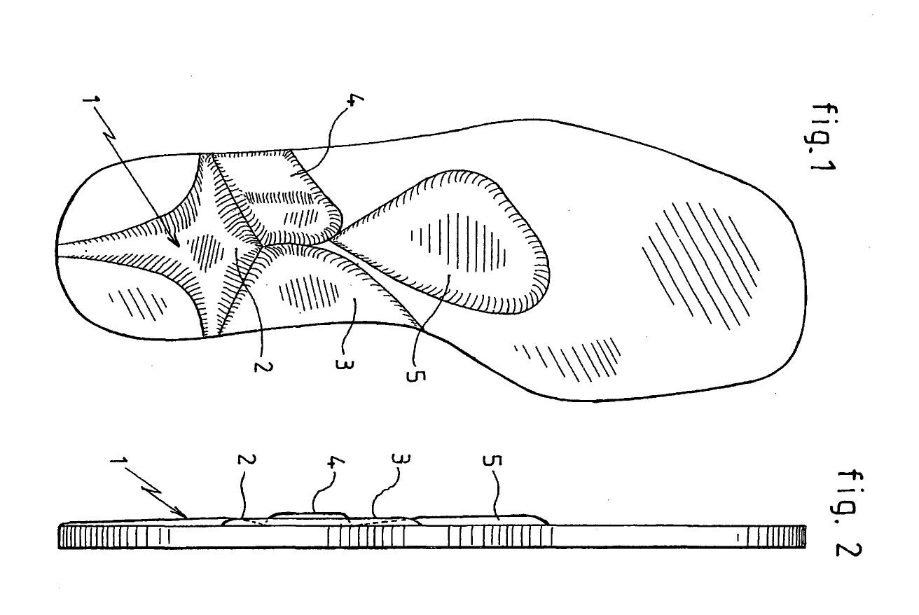

FIG. 1 is an overhead view of the orthopedic insole of the invention,

FIG. 2 is a side view of the orthopedic insole of the invention,

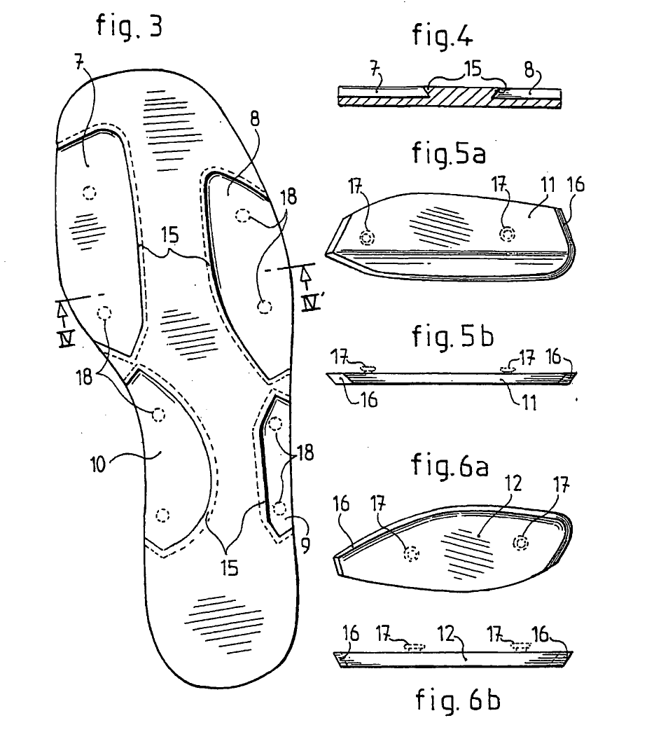

FIG. 3 is an underside view of the orthopedic insole of the invention,

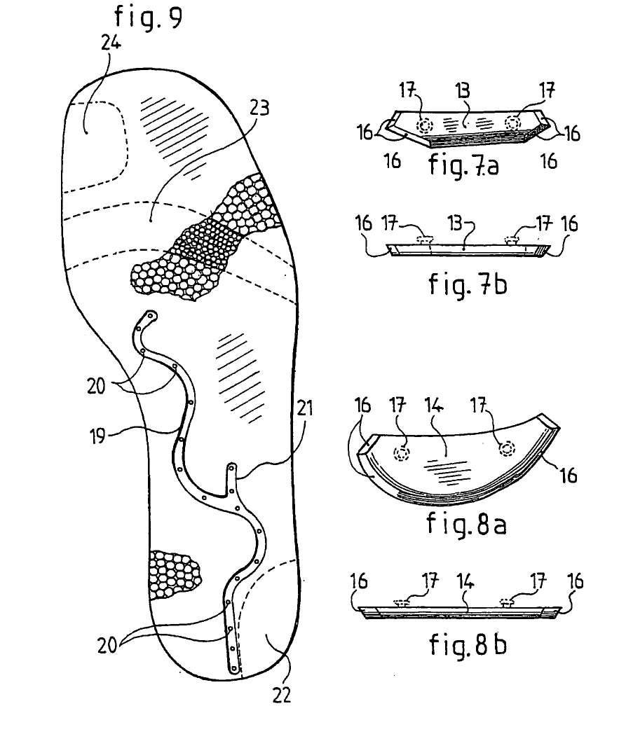

FIG. 4 is a cross-section view along axis IV-IV of the orthopedic insole shown FIG. 3,

FIGS. 5a to 8a are overhead views of the removable corrective elements of the orthopedic insole of the invention,

FIGS. 5b to 8b are side views of the removable corrective elements of the orthopedic insole shown FIGS. 5a to 8a,

FIG. 9 is an underside view of a variant of embodiment of this orthopedic insole of the invention.

For simplification reasons, only one insole of the invention will be described, corresponding to the left foot for example, the right foot being inferred by symmetry.

With reference to FIGS. 1 and 2, the upper surface of the insole comprises means for activating the articular receptors located between the talus and the calcaneus as soon as a step is habitually initiated by the heel and means for guiding the foot when it becomes engaged on the physiological axis of walking. These means essentially consist of a profiled channel, called console 1, and a set of profiled elements 3,4,5 distributed along the length of the insole to form a rail around which the foot is guided.

Console 1 extends longitudinally from the heel as far as the anterior end of the calcaneus, exactly perpendicular to the neck of the talus. Said console 1 increases in thickness from the heel as far as its anterior end 2. As a particular example, the height of console 1 gradually increases from 1 mm to 2 mm from the heel as far as its anterior end 2.

The set of profiled elements 3,4,5 from back to front, i.e. from the heel to the tip of the foot, consists of a sub-scaphoid profiled element 3, a sub-cuboid profiled element 4 and mediotarsal axial means 5. The sub-scaphoid profiled element 3 extends the console 1 towards the interior of the foot, semi-dome fashion. This sub-scaphoid profiled element 3 in this example has a height of approximately 2mm and thereby extends the anterior end 2 of console 1. The sub-cuboid profiled element 4, from an overhead view as in FIG. 1, is in the form of a kidney bean globally corresponding to the projection of the shape of the cuboid over the insole. This element 4 is positioned on the outer side of the sub-scaphoid element 3, its convexity facing backwardly at approximately 45° to the median longitudinal axis of the insole. The thickness of said element 4 increases from side to center and from rear to front and gradually reaches a height of around 4 mm. The mediotarsal means 5 have an obovate shape, i.e. roughly in the shape of a water drop, widening forwardly and ending just before the metatarsal heads of the foot. This mediotarsal element 5 is convex; its height varies longitudinally from a height of 2.5 mm to reach a maximum height in the order of 3.5 mm, at approximately two thirds of its length.

It will be noted that, when a step is taken, console 1 activates the calcaneus whether the foot is flat or arched, to prepare the remainder of the step under proper conditions; then the sub-scaphoid 3 and/or sub-cuboid 4 profiled elements, which act as side stabilizers for the foot, prompt the foot to remain within the physiological rail of walking and the mediotarsal element 5 prepares the final digitigrade phase of the step by distributing foot bearing under the metatarsal regions so that this bearing remains channeled along the axis of the second metatarsal through which the physiological axis of walking passes.

In addition, with reference to FIGS. 2 and 3, the insole on its undersurface comprises recesses 7,8,9 and 10 positioned along the inner and outer edge of the said insole and in which removable corrective elements 11, 12, 13 and 14 of shape corresponding to the shape of the recess are able to be positioned. The insole comprises a first recess 7 called an anti-abduction recess of substantially rectangular shape positioned along the inner edge of the insole and extending from the plantar arch as far as the great toe. The section of the anti-abduction recess 7, with reference to FIG. 3, increases from the inner edge of the insole in the direction of the median part of the insole over a short distance and then decreases. Also, with reference to FIG. 4, the wall of the anti-abduction recess 7 is inclined inwardly towards the inside of said recess 7 from its bottom part as far as the edge of said recess, i.e. as far as the undersurface of the insole so as to form a lip 15 on the edge of recess 7. This anti-abduction recess 7 is able to house an abduction corrective element 11 shown FIGS. 5a and 5b whose shape corresponds to the shape of the anti-abduction recess 7, i.e. of globally rectangular shape. This abduction corrective element 11 on its periphery comprises a chamfer 16 so that the lip 15 on the periphery of recess 7 maintains the removable element in place within said recess 7.

Evidently the edge of the abduction corrective element 11 which is adjacent to the inner edge of the insole when said element 11 is inserted inside anti-abduction recess 7 does not have a chamfer 16.

Also, the abduction corrective element 11 is made in a more rigid and denser material than the insole material and it is inserted into recess 7 by elastic deformation of lip 15. It will be noted that the particular shape of recess 7 and of the corresponding corrective element 11 prevents any undue displacement and also any egress of the said corrective element when walking or running. In addition, it will be observed that the corrective element does not come into contact with the foot, thereby avoiding any plantar skin lesion such as cuts, blisters, fissures or analog.

With reference to FIGS. 2 and 3, the insole also comprises a second recess, called an anti-adduction recess 8 of globally rectangular shape positioned along its outer edge and extending from the plantar arch as far as the cushion area of the small toe, a third recess called anti-varus or anti-supination recess 9 also of globally rectangular shape positioned along the outer edge of the insole and extending from the cuboid as far as the second anti-adduction recess 8, and a final recess called anti-valgus or anti-pronation recess 10 of semi-dome shape positioned along the inner edge of the insole under the plantar arch. In the same manner as previously, the walls of the anti-adduction 8, anti-varus 9 and anti-valgus 10 recesses are inclined inwardly towards the inside of said recesses 8, 9 and 10 from their bottom part as far as their respective edges so as to form a lip 15 on the edge of said recesses 8, 9 and 10. Each of the adduction 8, anti-varus 9 and anti-valgus 10 recesses is designed to house a corresponding corrective element for adduction 12, anti-varus 13 and anti-valgus 14 successively shown in FIGS. 6a, 6b, 7a, 7b and 8a, 8b; the adduction 12 and anti-varus 13 corrective elements are of globally rectangular shape and the anti-valgus corrective element 14 is of semi-domed shape. Each of these corrective elements is flat and made in a more rigid and denser material than the insole; these elements also comprise on their periphery a chamfer 16 to ensure their locking in position within their respective recesses.

According to one variant of embodiment of the insole of the invention, each of the corrective elements 11,12, 13 and 14 on their upper surface, i.e. the surface bearing upon the bottom of the recess, comprise two lugs 17 shown as dotted lines in FIGS. 5a to 8a and 5b to 8b, able to cooperate with two corresponding holes 18 positioned at the bottom of recesses 7, 8, 9 and 10; these corrective elements 11,12, 13 and 14 do not have a chamfer 16 and recesses 7, 8, 9 and 10 do not have a lip 15.

Evidently, the upper surface of the corrective elements may comprise any type of male of female attachment member able to cooperate with a respective male or female member positioned at the bottom of the recess to achieve attachment of the removable element within the recess, without departing from the scope of the invention.

Accessorily, with reference to FIG. 9, the insole on its under surface, comprises a furrow 19 of serpentine shape extending substantially from the forefoot as far as the heel and which comprises regularly distanced holes 20 leading to the upper surface of the insole. Furrow 19 comprises a small secondary branch 21 also provided with holes 20 and which extends substantially parallel to the median longitudinal axis of the insole under the plantar arch. It will be noted that this furrow 19 makes it possible to evacuate perspiration towards the bottom of shoe during intensive sports practice for example.

Also, the heel and forefoot of the insole advantageously have an alveolar structure, preferably honeycombed, to provide increased comfort. Since the foot's bearing intensity is greater in the posterior-external heel area, in the metatarsal head area and in the cushion area under the great toe when taking a step, the insole is provided in a posterior-external heel area 22 and/or metatarsal area 23 and/or cushion area under the great toe 24, with alveoli of smaller size than the alveoli in the remainder of the insole.

It will be noted that the height of the profiled elements positioned on the upper surface of the insole does not exceed a few millimetres so that the final insole is of average size suitable for all feet. Also it has been seen that, in surprising manner, the height or depth of the recesses positioned on the undersurface of the insole and of the removable elements arranged in at least one of the recesses does not exceed a few millimetres whilst providing proper correction of an abduction, adduction, varus and/or valgus.

Also, the removable corrective elements may be maintained in the recesses positioned on the undersurface of the insole by any appropriate means such as adhesive means or “Velcro” (registered trademark) without departing from the scope of the invention.

Finally, the orthopedic insole of the invention may evidently be made in any common rigid material that is slightly resilient either by molding or modeling. The aforesaid examples are also evidently only particular illustrations which are in no way restrictive in respect of the areas of application of the invention.

1. Modular proprioreceptive orthopedic insole to re-establish and/or preserve the correct dynamics of walking or running, said insole being made in an elastic material, characterized in that firstly on its upper surface it comprises means (1) for activating the articular receptors located between the talus and the calcaneus as soon as a step is habitually initiated by the heel, and means (3,4,5) for guiding the foot when it becomes engaged on the physiological axis of walking, and secondly on its undersurface it comprises at least one removable corrective element (11,12,13,14) able to provide abduction or adduction for respective deficient or excessive step initiation, and/or is able to correct a varus and/or valgus.

2. Orthopedic insole as in claim 1, characterized in that on its undersurface the insole comprises at least one recess (7,8,9,10) positioned along the inner and/or outer edge of said insole in which the removable corrective element (11 to 14) can be positioned that is made in a more rigid material than the insole material and whose shape corresponds to the shape of the recess (7 to 10).

3. Orthopedic insole as in claim 2 characterized in that the section of the recess (7 to 10) increases from the inner or outer edge of the insole in the direction of the median part of the insole over a short distance and then decreases.

4. Orthopedic insole as in claim 2, characterized in that the wall of the recess (7 to 10) is inclined inwardly towards the inside of said recess from the bottom part as far as its edge to form a lip (15) on the edge of the recess (7 to 10), and in that the removable element (11 to 14) on its periphery comprises a chamfer (16) so that the lip (15) holds the removable element in position within the recess (7 to 10), said element being inserted in the recess by elastic deformation of the lip (15).

5. Orthopedic insole as in claim 2, characterized in that the removable element (11 to 14) on one of its surfaces comprises a male attachment member (17) able to cooperate with a female member (18) positioned at the bottom of the recess (7 to 10) to achieve attachment of the removable element within the recess.

6. Orthopedic insole as in claim 2, characterized in that it comprises an anti-abduction recess (7) positioned along the inner edge of the insole and extending from the plantar arch as far as the great toe.

7. Orthopedic insole as in claim 1, characterized in that it comprises an anti-adduction recess (8) positioned along the outer edge of the insole and extending from the plantar arch as far as the cushion area of the small toe.

8. Orthopedic insole as in claim 2, characterized in that it comprises an anti-valgus or anti-pronation recess (10) positioned along the inner edge of the insole under the plantar arch.

9. Orthopedic insole as in claim 2, characterized in that it comprises an anti-varus or anti-supination recess (9) positioned along the outer edge of the insole and extending from the cuboid as far as the second anti-adduction recess (8).

10. Orthopedic insole as in claim 1, characterized in that on its upper surface it is provided with a sub-calcaneus longitudinal profiled channel, or console (1), whose thickness increases from the heel as far as an area located substantially perpendicular to the neck of the talus.

11. Orthopedic insole as in claim 10, characterized in that on its upper surface it comprises a sub-scaphoid profiled element (3) of substantially semi-domed shape extending the console (1) towards the inside of the foot.

12. Orthopedic insole as in claim 10, characterized in that on its upper surface it comprises a sub-cuboid element (4) positioned on the outer side of the sub-scaphoid element (3) and in the shape of a kidney bean, having its convexity facing backwardly at approximately 45° to the median longitudinal axis of the insole.

13. Orthopedic insole as in claim 11, characterized in that on its upper surface it comprises mediotarsal axial means (5) of obovate shape widening forwardly and ending just in front of the metatarsal heads so as to distribute bearing under the metatarsal regions.

14. Orthopedic insole as in claim 1, characterized in that on its undersurface it comprises a furrow (19) of serpentine shape extending substantially from the flat of the foot as far as the heel and which comprises regularly distanced holes (20) leading to the upper surface of the insole so as to allow the evacuation of perspiration.

15. Orthopedic insole as in claim 1, characterized in that the heel and the forefoot of the insole have an alveolar structure.

16. Orthopedic insole as in claim 15, characterized in that the alveolar structure consists of a honeycomb structure.

17. Orthopedic insole as in claim 15, characterized in that in a posterior-external heel area and/or metatarsal head area (23) and/or in a cushion area under the great toe (24) it comprises alveoli of smaller size than the alveoli in the remainder of the insole.

18. Orthopedic insole as in claim 1, characterized in that the height or depth of the profiled elements (1,3,4,5) positioned on the upper surface of the insole, of the recesses (7 to 10) positioned on the undersurface of said insole, and of the removable elements (11 to 14) arranged in at least one of the recesses, does not exceed a few millimetres.

19. Orthopedic insole as in claim 3, characterized in that the wall of the recess (7 to 10) is inclined inwardly towards the inside of said recess from the bottom part as far as its edge to form a lip (15) on the edge of the recess (7 to 10), and in that the removable element (11 to 14) on its periphery comprises a chamfer (16) so that the lip (15) holds the removable element in position within the recess (7 to 10), said element being inserted in the recess by elastic deformation of the lip (15).

20. Orthopedic insole as in claim 3, characterized in that the removable element (11 to 14) on one of its surfaces comprises a male attachment member (17) able to cooperate with a female member (18) positioned at the bottom of the recess (7 to 10) to achieve attachment of the removable element within the recess.
